(19) United States
(12) Patent Application Publication
(10) Pub. No.: US 2011/0039322 A1
(43) Pub. Date: Feb. 17, 2011

(54) METHOD FOR PRODUCING BIOGAS

(76) Inventor: Urs Inauen, Appenzell (CH)

Correspondence Address: Browdy and Neimark, PLLC, 1625 K Street, N.W., Suite 1100, Washington, DC, 20006, US

(21) Appl. No.: 12/680,002

(22) PCT Filed: Sep. 22, 2008
(86) PCT No.: PCT/EP2008/062627

§ 371 Date: Aug. 10, 2010

(30) Foreign Application Priority Data
Sep. 25, 2007 (CH) 01485/07

(51) Int. Cl. C12P 5/02; C12M 3/02

(52) U.S. Cl. 435/167; 435/303.3

(57) ABSTRACT

The invention relates to a method for producing biogas, wherein biomass (B) is filled into a biogas reactor (R), and a plurality of mixing bodies (1) is disposed on the biomass (B). The mixing bodies (1) float on the surface (O) of the biomass (B) and thereby dip partially into the biomass (B). The surface of the biomass is thus mixed, leading to an increase in the efficiency of gas yield.

### TECHNICAL FIELD OF THE INVENTION

The present invention relates to a method for producing biogas according to the features of the preamble of claim 1 and to mixing bodies configured especially for use in such a method.

### PRIOR ART

Biogas reactors, fermenting tuns or fermenting tanks are known from the prior art. The reactors are filled with fermentable biomass, such as residual substances (clarify sludge, biowaste, food remains), manure (liquid manure or dung), or with other suitable materials. Fermentable biomass or fermentable materials may have a liquid and/or a solid state. As a result of the fermentation of said materials, a biogas can be obtained, which may be used, for example, for driving gas generators or motor vehicles or for combustion in a heating system.

Such a reactor is usually in the form of a cylindrical container which is closed by means of a cover. The reactor is filled with biomass, from which the biogas can then be obtained. In this case, the biogas flows out of the biomass upward where it can be extracted from the reactor in the region of the cover. Furthermore, such a reactor comprises an agitating mechanism which constantly stirs around the fermentable materials. Moreover, heating elements may be provided, which additionally accelerate or assist the heating of the biomass and fermentation.

The liquid constituents of the fermentable materials are usually located in the region of the bottom, and solid constituents of lower density float on these liquid constituents. This floating layer is detrimental to the efficiency of fermentation or gas formation. In devices of the prior art, therefore, vane-type agitating mechanisms have been provided which partially break through this floating layer. However, the effect of such a partial breakthrough is often unsatisfactory, since the floating layer is repeatedly reformed.

### PRESENTATION OF THE INVENTION

Proceeding from this prior art, one object of the invention is to specify a method which improves the efficiency of biogas reactors. Furthermore, a method is to be specified which can be employed in a simple way in already existing biogas reactors.

This object is achieved by means of a method for producing biogas having the features of patent claim 1.

A further object is to specify a mixing body designed especially for use in such a method. This object is achieved by means of a mixing body having the features of patent claim 6 or 10.

Accordingly, in a method for producing biogas, in which biomass is introduced into a biogas reactor, a multiplicity of mixing bodies are arranged on the biomass. The mixing bodies float on the surface of the biomass and at the same time dip partially into the biomass.

Preferably, furthermore, a mixing device is present, which is suitable for fully mixing the biomass actively, for example a conventional agitating mechanism, or the biomass is set in motion in another way. Due to the presence of the mixing bodies, the surface of the biomass in the biogas reactor is then fully mixed especially efficiently, since, because the biomass is in motion, the hollow bodies are likewise set in motion. In particular, the formation of a floating layer on the biomass is prevented. This has a positive influence upon the efficiency of gas recovery in a biogas reactor. Furthermore, the surface of the biomass is additionally increased, thus likewise influencing the efficiency in a positive way.

Preferably, the mixing bodies cover between 20% and 85%, in particular 30% and 70%, of the surface of the biomass.

Preferably, the mixing bodies are designed essentially as hollow bodies.

Preferably, the mixing bodies have a surface structure. In this case, the full-mixing process is even more effective, since the biomass which is in motion can engage especially well on the surface structure, and therefore a good full mixing of the biomass is ensured.

Preferably, the mixing bodies are of essentially spherical design. The advantage of this is that the mixing bodies are effectively comoved due to the movement of the biomass and can easily rotate about any axes.

The mixing body is preferably designed as a hollow body with at least one orifice, the at least one orifice allowing entry and exit of biomass into and out of the hollow body. Alternatively, the hollow body may also be designed without an orifice, in which case this hollow body may also be designated as a closed hollow body.

The biomass entering the hollow body through the orifice can likewise ferment in the hollow body. Moreover, when the hollow body rotates, parts of the biomass adhere to the wall, thus additionally accelerating the fermentation.

Preferably, the hollow body comprises at least one further orifice. As a result, more biomass can enter the hollow body within the same period of time, thus further increasing the efficiency.

Preferably, the further orifice is arranged essentially diametrically opposite the first orifice, with the result that the mixing body is well balanced.

Preferably, the mixing body has at least one mixing element which is arranged on the outwardly facing surface of the wall and which projects from this. The mixing element ensures a better full mixing of the biomass or separation of a possible floating layer and acts at the same time as a driver element, so that the mixing body can be effectively driven by the moving biomass. The mixing element can be attached both to the closed mixing body or hollow body and to the mixing body or hollow body having at least one orifice.

Preferably, the mixing body comprises at least one buoyancy body which prevents the situation where the mixing device may sink completely. Moreover, the mixing body is always oriented in the biomass preferably so that parts of at least one orifice dip into the biomass. This ensures that the biomass can penetrate into the hollow body.

Preferably, the at least one buoyancy body is arranged inside the hollow body. The at least one buoyancy body may be present both in the closed mixing body or hollow body and in the hollow body having at least one orifice. In addition, in a version with a buoyancy body, one or more mixing elements may also be arranged on the surface of the mixing body.

The at least one buoyancy body preferably has a cavity filled with a medium which has a lower density than the biomass.

Preferably, the medium with which the at least one buoyancy body is filled is a gas, in particular air, or the buoyancy body may be manufactured from a foamed plastic.

Preferably, the at least one buoyancy body is formed in one piece with the wall of the hollow body, in which case the hollow body can then be produced especially efficiently and cost-effectively.

Further advantageous embodiments are specified in the subclaims.

### BRIEF DESCRIPTION OF THE DRAWING

Preferred embodiments are described in more detail below, by way of example, with reference to the drawing in which:

FIG. 1 shows a diagrammatic top view from above into a biogas reactor which is equipped with mixing bodies according to the invention;

FIG. 2 shows a diagrammatic view of a detail of a biogas reactor from the side;

FIG. 3a shows a diagrammatic top view of a mixing body according to the invention in a first exemplary embodiment;

FIG. 3b shows a sectional illustration of the mixing body according to FIG. 3a;

FIG. 3c shows a front view of the mixing body according to FIG. 3a;

FIG. 4a shows a diagrammatic top view of a mixing body according to the invention in a second exemplary embodiment;

FIG. 4b shows a sectional illustration of the mixing body according to FIG. 4a; and

FIG. 4c shows a front view of the mixing body according to FIG. 4a.

### DESCRIPTION OF PREFERRED EXEMPLARY EMBODIMENTS

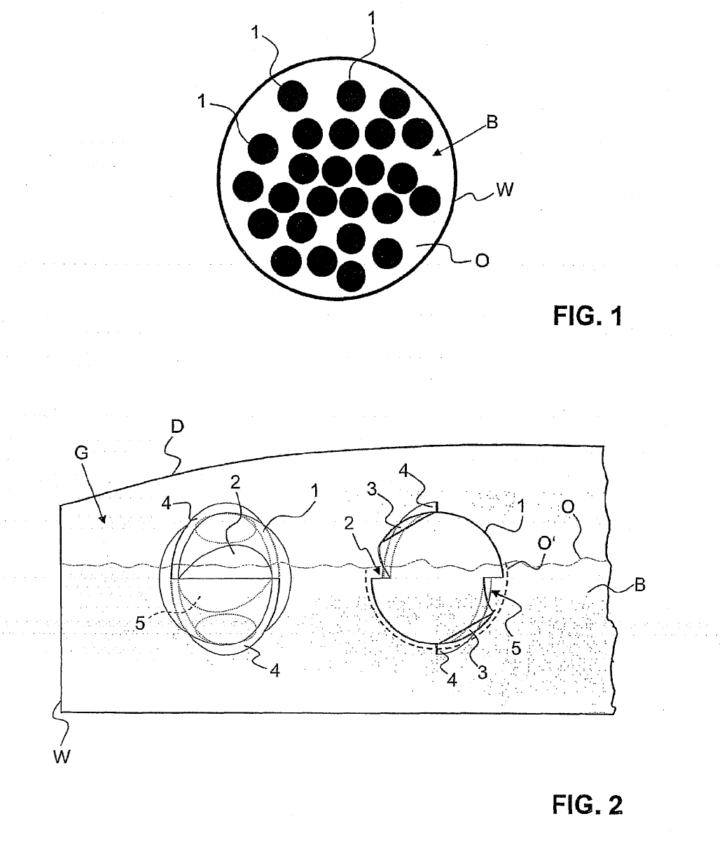

FIG. 1 shows, in a diagrammatic illustration, a top view of the inside of a biogas reactor or of a fermenting tun. Typically, such biogas reactors have surfaces of several 100 m². The biogas reactor is illustrated merely diagrammatically here and is delimited essentially by a cylindrical side wall W. The biogas reactor is filled with biomass B. A multiplicity of mixing bodies 1 are arranged on the surface ◯ of the biomass B in order to improve the efficiency of a biogas reactor and partially dip into this biomass. Preferably, so many mixing bodies 1 are put in place that the mixing bodies 1 cover between 20% and 85%, in particular 30% and 70%, of the surface ◯ of the biomass B.

FIG. 2 shows a detail of said biogas reactor in a side view with two mixing bodies 1. It is indicated diagrammatically here that, furthermore, the biogas reactor has a cover D. The biomass B is located essentially in the lower region of the biogas reactor, while a gas mixture G of biogas and/or air is located in the upper region. This gas mixture can be extracted from the biogas reactor by means of an extraction point, not shown. The mixing body 1 illustrated on the right side is depicted as a sectional illustration, while that illustrated on the left side is depicted as a front view. The mixing body 1 is designed essentially as a hollow body. The hollow body has a wall 10 which delimits a cavity 11 for the reception of air, biogas and/or biomass, and may be of differing design. The hollow body is configured so that it floats on the biomass B or dips partially into this. Furthermore, the hollow body may be equipped with at least one orifice 2.

As a result of the movement of an agitating mechanism, the entire biomass B is set in motion. On account of the resulting flow, the mixing bodies 1 are also set in motion on the biomass. Due to the motion, the mixing bodies 1 rotate about their own axes, thus leading to a breakthrough or full mixing of the floating layer on the biomass B. Due to the motion, biomass B or parts of the gas mixture G can also repeatedly enter the hollow body and re-emerge from this. The full mixing of the biomass B is thereby accelerated. Furthermore, the biomass B can also ferment in the hollow sphere.

Owing to the presence of the freely moving mixing bodies 1, an efficient full mixing of the entire surface of the biomass B takes place. By the mixing bodies 1 being provided, the occurrence of a floating layer on the surface of the biomass is prevented. If a floating layer nevertheless unexpectedly forms, this floating layer which occurs is constantly split up by the mixing bodies 1. The fermentation process can thereby be influenced positively, thus leading to an increase in the efficiency of gas recovery. This is an advantage, as compared with devices of the prior art, in particular as compared with straightforward vane-type agitating mechanisms, in which the floating layer is broken through only at local points.

Furthermore, the presence of the mixing bodies 1 brings about an increase in the surface O of the biomass B, with the result that the fermentation of the biomass B is positively influenced or accelerated. The increase in the surface O is brought about in that the hollow body 1 dips or projects partially into the biomass B and, during a rotational movement, repeatedly drives biomass B on its surface. The surface of the biomass B in the absence of the mixing bodies 1 corresponds essentially to the projection of the mixing body 1. The surface O which is afforded by the presence of the mixing body corresponds to that surface of the hollow body which is dipped into the biomass. An increased surface O is thus achieved as a result of the arrangement.

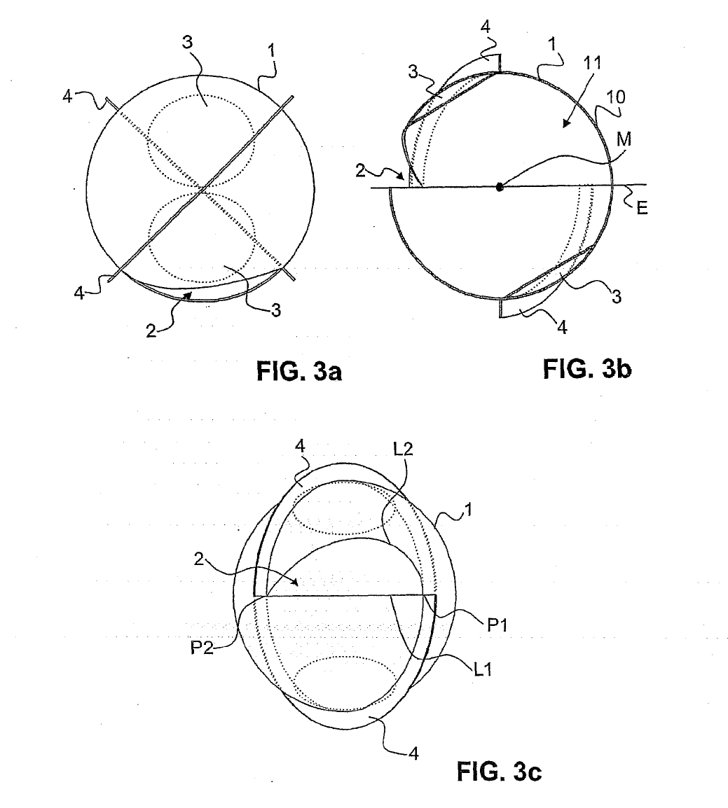

FIGS. 3a to 3c show a mixing body 1 according to a first exemplary embodiment. The mixing body 1 has here essentially the form of a hollow sphere. Due to the presence of the orifice 2, biomass can enter the hollow body 1 and re-emerge from it through the orifice 2. This is assisted by the form of the orifice 2, since a kind of scooping movement occurs as a result of the rotation of the hollow body 1. Since the hollow body 1 rotates continuously, the cavity 11 of the hollow body can be filled partially with biomass. The biomass located in the cavity 1 likewise ferments, the result of which is that the cavity 11 also contains part of the gas mixture G or biogas. During further movements of the hollow body 1, this part of the biogas or the fermenting biomass is intermixed again into the biomass B which is located in the bioreactor. Fermentation is thereby accelerated.

The orifice 2 extends over a defined surface portion and is delimited by a first line L1 and a second line L2. The first line L1 is the line of intersection on the surface of the hollow body 1, here of the hollow sphere 1, with a plane E which runs through the mid-point M of the hollow sphere 1. That is to say, in other words, the line L1 runs equatorially. In this case, the line L1 extends from a starting point P1 to an end point P2. While this is advantageous for the reception of biomass, the sectional plane may also extend above or below the mid-point. The second line L2 extends on the surface of the hollow body 1, here of the hollow sphere 1, arcuately or curvedly from the starting point P1 of the line L1 to the end point P2 of the line L2.

Moreover, the mixing body 1 comprises at least one buoyancy body 3. In the present exemplary embodiment, two buoyancy bodies 3 are arranged which, here, are placed on the inside of the wall 10. The arrangement of the buoyancy bodies 3 prevents the mixing bodies 1 from sinking when these are filled with biomass B. Preferably, one of the buoyancy bodies 3 is arranged adjacently to the orifice 2. In the present exemplary embodiment, the buoyancy body is arranged in the region of the arcuate line L2. The other buoyancy body 3 is arranged diametrically opposite the first buoyancy body. In the state of rest, the buoyancy body lies, oriented, in the biomass B in such a way that both buoyancy bodies dip into the biomass B. This orientation also ensures that at least one orifice 2 is located at least partially in the biomass B, so that the biomass can enter the hollow body. On account of the motion of the biomass, the mixing body 1 rotates essentially about the axis which extends from the mid-point of one hollow body to the mid-point of the other hollow body. However, on account of the biomass B in the cavity, a kind of wobbling motion may also result. Any desired mixed forms of motion may also be envisaged.

The buoyancy body 3 has the form of a hollow body which is filled with a substance, the density of which is lower than the density of the biomass. In particular, the substance may be air or another gas. Instead of a hollow body, a buoyancy body consisting of a plastic, in particular of a foamed plastic, such as polystyrene, may also be provided. The arrangement of a buoyancy body prevents the situation where the mixing body 1 may sink in the biomass B.

Alternatively, the hollow body 1 itself may also provide sufficiently high buoyancy. For this purpose, the hollow body 1 is manufactured from a material which gives rise to buoyancy even when the hollow body 1 is filled with biomass. For example, it is conceivable to manufacture the entire hollow body 1 from a foamed plastic, such as polystyrene.

In the present exemplary embodiment, the mixing body 1 comprises at least one tail-like or comb-like mixing element 4. This mixing element 4 is arranged on the outside of the wall 10 and projects from the wall 10. The mixing element 4 illustrated here is a sheet-like element which extends over part of the surface of the mixing body 1. The mixing element 4 not only has a mixing function, in which a possible floating layer on the surface is separated, but also a driving function or propelling function. Since the mixing elements 4 project above the surface of the mixing body 1, the flow of biomass B can engage on the mixing elements 4, the rotational movement of the mixing body 1 being further assisted. Furthermore, these mixing elements 4 separate a possible floating layer. Due to both effects, the efficiency or the recovery of biogas from the biomass can be further increased.

In the present exemplary embodiment, a first mixing element 4 extends on the top side of the hollow body 1 along a first meridian and a second mixing element 4 extends on the underside of the hollow body 1 along a second meridian. The equatorial plane E in this case serves as a parting plane between the underside and top side and penetrates through the sphere through the mid-point of the latter. A meridian is understood to mean a line which extends perpendicularly from the equatorial plane on the sphere surface to one of the poles. The first meridian is arranged at an angle to the second meridian in the equatorial plane E. Preferably, the angle of the first meridian to the second meridian is 45° to 135°. The exemplary embodiment according to FIG. 3a-3c shows an arrangement in which the meridians are offset at 90° with respect to one another. Accordingly, a first mixing element 4 extends along the first meridian over the surface of the top side of the mixing body 1 from a starting point which coincides with the point P2 to an end point which lies diametrically opposite P2. A further mixing element 4 extends over the surface of the underside of the mixing body 1 along the second meridian from a starting point which coincides with the point P1 to an end point which lies diametrically opposite P1. Alternatively, the mixing elements 4 may also extend along any curve on the surface of the mixing body 1.

In an alternative embodiment, that surface of the wall 10 which faces the biomass B may be provided with a surface structure which serves for driving the hollow body 1 and/or for the full mixing of the biomass B. The biomass B can thereby be fully mixed especially effectively, since, during a rotation of the hollow body 1, parts of the biomass B dwell on the surface. The biomass dwelling on the surface of the hollow body 1 is in this case exposed to the air or the biogas and can ferment. For example, a roughened surface structure may be envisaged. In another embodiment, moreover, it is conceivable that a multiplicity of spike-like mixing elements project from the surface of the hollow body. Furthermore, it is also conceivable to combine the surface structures described with the sheet-like mixing element 4.

Furthermore, it is also conceivable to provide that surface of the wall 10 which faces the cavity 11 with a corresponding surface structure as well. As a result, the biomass which is located in the hollow body can likewise be fully mixed effectively.

The hollow sphere 1 typically has a diameter in the range of 20 cm to 180 cm, in particular of between cm and 150 cm. If the hollow body 1 has another form, for example is oval, elliptically rectangular or square in cross section, these dimensions relate to the maximum extent of the hollow body.

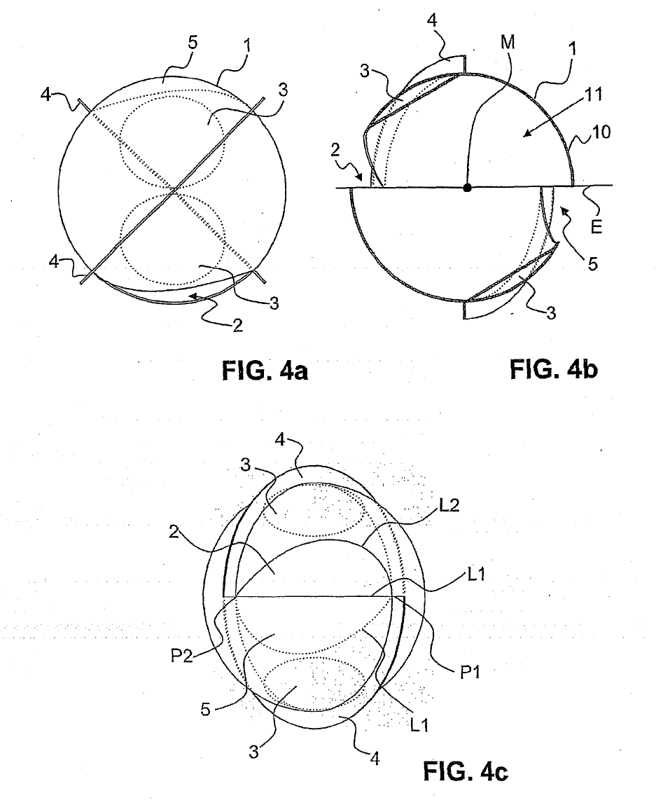

FIGS. 4a to 4c show a second embodiment of the mixing body 1 according to the invention. Identical parts are designated by the same reference symbols. Furthermore, part features of all the exemplary embodiments described may be combined with one another, as desired, to form further exemplary embodiments.

The second embodiment of the mixing body 1, which is likewise designed here as a hollow sphere, comprises a further orifice 5. Preferably, the further orifice 5 is arranged diametrically to the first orifice 2. That is to say, in other words, the first orifice 2 extends from the plane E upward, and the second orifice 5 extends from the plane E downward. Furthermore, the further orifice 5 is preferably designed with an identical form and dimensions to the first orifice 2. By a further orifice 5 being arranged, a larger quantity of biomass B can flow into the cavity and leave this over an identical period of time. The effectiveness of the mixing body according to the invention can thereby be increased further.

In further embodiments, moreover, further orifices may be arranged. Alternatively, it is also possible to provide a mixing body or hollow body 1 which has no orifices. A closed mixing body is in this case referred to. Such a mixing body may be designed to be hollow or may be filled with a material which has a lower density than the biomass, so that the mixing body floats on the biomass. Alternatively, moreover, such a mixing body may comprise a mixing element 4, as described above.

Preferably, the mixing body or hollow body 1 is manufactured from a plastic which is resistant to the biomass and to the biogases resulting from the biomass. Examples of a plastic are: polypropylene, polyethylene or HDPE, acrylonitrile-butadiene-styrene, etc.

The mixing body 1 according to the invention may be produced, for example, by means of an injection molding method. For this purpose, the mixing body 1 may also be formed from two parts or half shells, to be precise from an upper part, which extends above the plane E, and a lower part, which extends below the plane E. The two parts can then be connected, in particular adhesively bonded or welded, to one another. Alternatively, the entire mixing body 1 may also be produced in one piece.

In an alternative embodiment, the mixing body 1 may also be manufactured from stainless steel or aluminum, the surface of which is optionally anodized, or from other metallic materials. Here, too, the mixing body 1 may be manufactured, for example, from two half shells, as described above, which are then connected to one another.

### LIST OF REFERENCE SYMBOLS

1 Mixing body, hollow body

10 Wall

11 Cavity

2 First orifice

3 Buoyancy body

4 Mixing element

5 Second orifice

L1 First line

L2 Second line

P1 Starting point

P2 End point

W Side wall

D Cover

B Biomass

G Gas mixture

O Surface of the biomass

M Mid-point

E Plane

1-18. (canceled)

19. A biogas reactor for producing biogas, wherein said biogas reactor comprising biomass and a multiplicity of mixing bodies wherein, said mixing bodies float on the surface of the biomass and at the same time dip partially into the biomass.

20. The biogas reactor as claimed in claim 19, wherein the mixing body further comprising:
a wall providing a hollow body; and
at least one orifice which is arranged in said wall,
wherein said at least one orifice is adapted to allow biomass to enter into and to exit out of the hollow body.

21. The biogas reactor as claimed in claim 20, wherein the hollow body has at least one further orifice.

22. The biogas reactor as claimed in claim 20, wherein the hollow body has at least one further orifice which is arranged diametrically opposite the first orifice.

23. The biogas reactor as claimed in claim 20, wherein the orifices extend over a surface portion which is delimited by a first line and a second line, wherein the first line being a line of intersection on the surface of the hollow body with a plane, wherein the first line extending from a starting point to an end point, and the second line being an arcuate line which extends on the surface of the hollow body from said starting point to said end point of the first line.

24. The biogas reactor as claimed in claim 19, wherein the mixing body further comprising:
an outer surface, and
at least one mixing element which is arranged an the outer surface of said wall.

25. The biogas reactor as claimed in claim 24, wherein the mixing body has a spherical basic form, and wherein a first elongate mixing element extends an the top side of the hollow body along a first meridian, and wherein a second elongate mixing element extends an the underside of the hollow body along a second meridian, the first mixing element and the second mixing element being arranged. Offset at an angle with respect to one another, in the equatorial plane.

26. The biogas reactor as claimed in claim 19, wherein the mixing body further comprising:
a wall having an outer surface said wall providing a hollow body;
at least one orifice which is arranged in said wall; and
at least one mixing element which is arranged an said outer surface of the wall,
wherein said at least one orifice is adapted to allow biomass to enter into and to exit out of the hollow body.

27. The biogas reactor as claimed in claim 26, wherein the hollow body has at least one further orifice which is arranged diametrically opposite the first orifice.

28. The biogas reactor as claimed in claim 26, wherein the orifices extend over a surface portion which is delimited by a first line and a second line, wherein the first line being a line of intersection an the surface of the hollow body with a plane, wherein the first line extending from a starting point to an end point, and the second line being an arcuate line which extends an the surface of the hollow body from said starting point to said end point of the first line.

29. The biogas reactor as claimed in claim 26, wherein the mixing body has a spherical basic form, and wherein a first elongate mixing element extends an the top side of the hollow body along a first meridian, and wherein a second elongate mixing element extends an the underside of the hollow body along a second meridian, the first mixing element and the second mixing element being arranged, offset at an angle with respect to one another, in the equatorial plane.

30. The biogas reactor as claimed in claim 26, wherein the angle between the two mixing elements in the equatorial plane is between 45° and 135°.

31. The biogas reactor as claimed in claim 20, wherein the mixing body further comprises at least one buoyancy body.

32. The biogas reactor as claimed in claim 31, wherein the at least one buoyancy body is arranged inside said mixing body.

33. The biogas reactor as claimed in claim 31, wherein the at least one buoyancy body has a cavity filled with a medium which has a lower density than the Biomass.

34. The biogas reactor as claimed in claim 31, wherein the medium with which the at least one buoyancy body is filled is a gas, air, or a foamed plastic.

35. The biogas reactor as claimed in claim 31, wherein the at least one buoyancy body is formed in one piece with the wall of the hollow body.

36. The biogas reactor as claimed in claim 26, wherein the mixing body further comprises at least one buoyancy body.

37. A method for producing biogas wherein
biomass is introduced into a biogas reactor, and
a multiplicity of mixing bodies are arranged on the biomass, which float on the surface of the biomass and at the same time dip partially into the biomass.

38. The method for producing biogas as claimed in claim 37, wherein the mixing bodies cover between 20% and 85% of the surface of the biomass.

39. The method for producing biogas as claimed in claim 37, wherein the mixing bodies cover between 30% and 70% of the surface of the biomass.

40. The method for producing biogas as claimed in claim 37, wherein the mixing bodies are designed as hollow bodies.

41. The method as claimed in claim 37, wherein the mixing bodies have a surface structure.

42. The method as claimed in claim 37, wherein the mixing bodies are spherical.

43. A mixing body for use in a method as claimed in claim 37, wherein the mixing body having
a wall providing a hollow body; and
at least one orifice which is arranged in said wall, wherein said at least one orifice is adapted to allow biomass to enter into and to exit out of the hollow body.

44. The mixing body as claimed in claim 43, wherein the hollow body has at least one further orifice.

45. The mixing body as claimed in claim 43, wherein the orifices extend over a surface portion which is delimited by a first line and a second line, wherein the first line being a line of intersection on the surface of the hollow body with a plane, wherein the first line extending from a starting point to an end point, and the second line being an arcuate line which extends on the surface of the hollow body from said starting point to said end point of the first line.

46. A mixing body for use in a method as claimed in claim 37, wherein the mixing body has at least one mixing element which is arranged on the outside of the mixing bodies.

47. The mixing body as claimed in claim 46, wherein the mixing body has a spherical basic form, and wherein a first elongate mixing element extends on the top side of the hollow body along a first meridian, and wherein a second elongate mixing element extends on the underside of the hollow body along a second meridian, the first mixing element and the second mixing element being arranged, offset at an angle with respect to one another, in the equatorial plane.

48. The mixing body as claimed in claim 47, wherein the angle between the two mixing elements in the equatorial plane is between 45° and 135°.

49. The Biogas reactor as claimed in claim 43, wherein the at least one buoyancy body is arranged inside said mixing body.
